(19) United States
(12) Patent Application Publication
(10) Pub. No.: US 2010/0114265 A1
(43) Pub. Date: May 6, 2010

(54) DEVICE FOR IRRADIATING AN OBJECT, IN PARTICULAR THE HUMAN SKIN, WITH UV LIGHT

(76) Inventor: Andreas LECHTHALER, Nenzing (AT)

Correspondence Address: WENDEROTH, LIND & PONACK, L.L.P., 1030 15th Street, N.W.,, Suite 400 East, Washington, DC, 20005-1503, US

(21) Appl. No.: 12/636,085

(22) Filed: Dec. 11, 2009

Related U.S. Application Data
(63) Continuation of application No. PCT/AT2008/000204, filed on Jun. 12, 2008.

(30) Foreign Application Priority Data
Jun. 13, 2007 (AT) A 915/2007

(51) Int. Cl. A61N 5/06

(52) U.S. Cl. 607/94

(57) ABSTRACT

The invention relates to a device for irradiating an object, in particular human skin, with UV light. Said device comprises a UV light source and an irradiation head containing imaging optics, UV light being projected from the irradiation head onto the object. According to the invention, a position detection unit is provided for the contactless detection of the spatial progression of the region on the surface of the object that is to be irradiated.

The invention concerns a device for irradiating an object, in particular the human skin, with UV light, comprising a UV light source and an irradiation head which includes an optical imaging system and from which UV light is projected on to the object.

In irradiation with UV light, besides the amount of energy delivered by the UV light source or the irradiation head and the duration of the irradiation procedure, the position and in particular the spacing of the object to be irradiated from the irradiation head is also an important consideration.

To be able to irradiate an object with an exactly defined level of radiation intensity in a substantially automated and precise irradiation process, the invention provides that there is provided a position detection device for contactless detection of the spatial configuration of the region of the surface of the object, that is to be irradiated.

By way of such a position detection device, it is possible to establish the surface to be irradiated or a desired surface portion exactly and in automated fashion (even when the object is moving) and thus to correspondingly adapt the radiation dose delivered by the irradiation head.

In that respect it is possible to use a position detection device in the form of a distance camera which measures the distance on the basis of the time-of-flight principle (TOF principle). That involves an electrooptical measurement process. In that case electronic components (such as for example a CMOS-CCD) record the high-frequency infrared radiation reflected by an object to be detected. A logic evaluation system compares the phase position of emitted and received light and on the basis of the speed of light calculates the distance covered by the light beam.

In accordance with a preferred embodiment it can therefore be provided that the position detection device includes a TOF camera for detecting the surface geometry and the spacing relative to the surface of the object. In particular in that case during irradiation with UV light by the irradiation head, the TOF camera is intended to measure the surface geometry and the spacing relative to the surface. It is thus possible to directly adapt the dosage of the UV light in dependence on the calculated distance or distances covered. If therefore the body for example moves partially away from the irradiation head during the treatment (thus for example upon exhalation), the intensity on the region of the body that is moving away is correspondingly increased and adapted to the respective movement.

Further advantages and details of the invention are set forth in the specific description hereinafter.

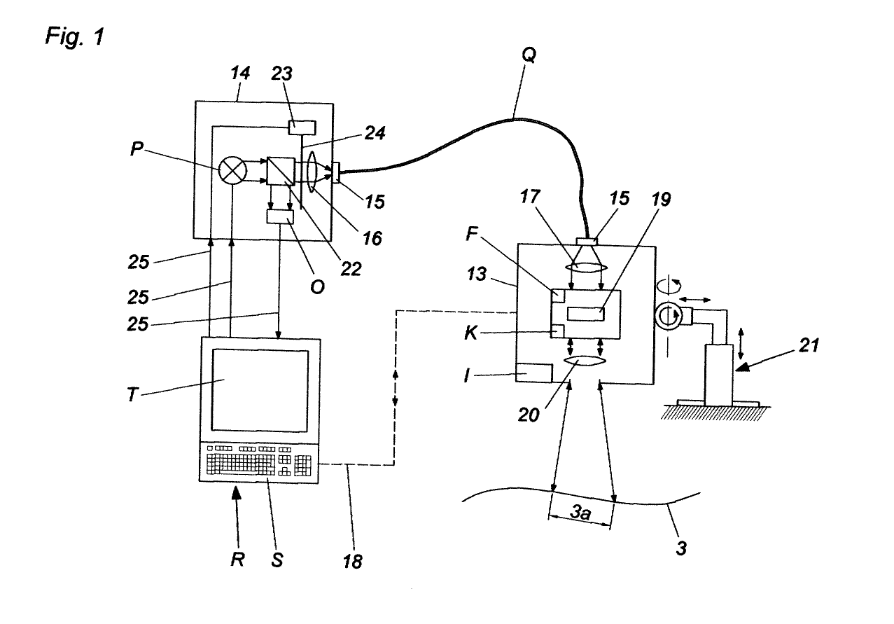

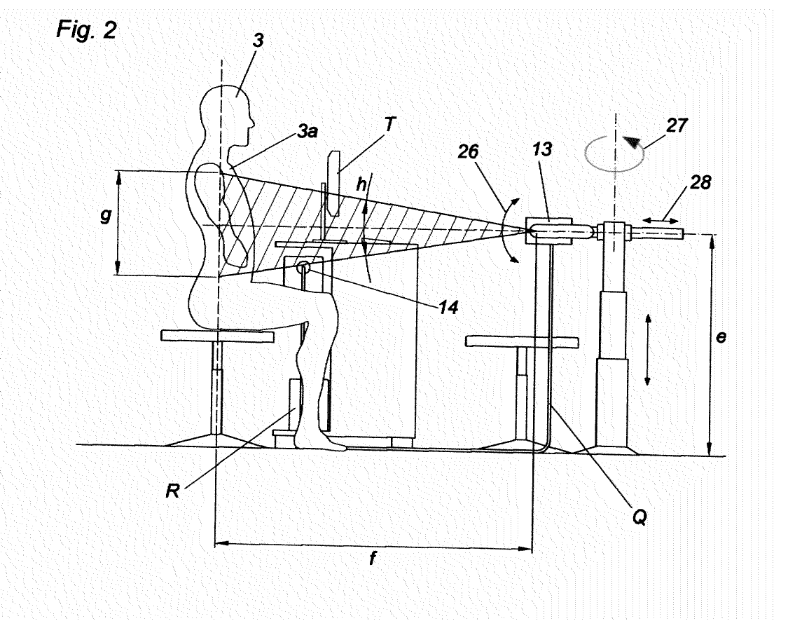

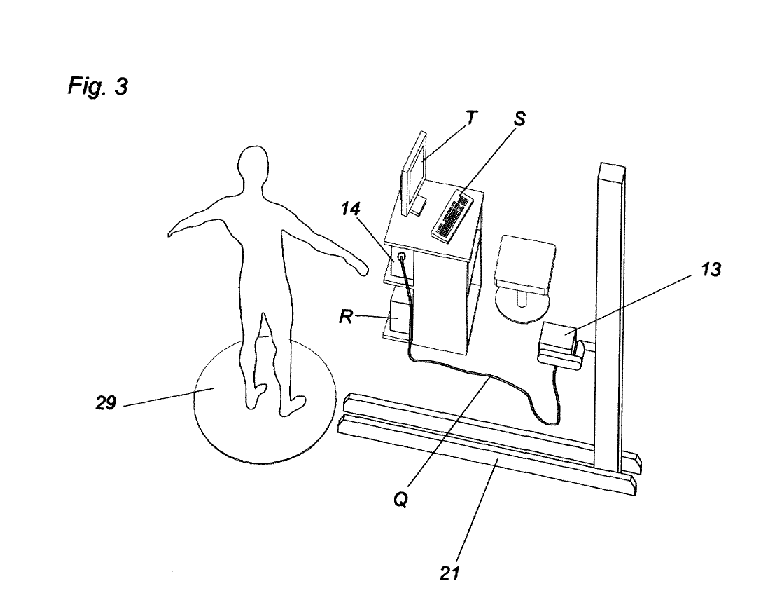

FIG. 1 shows a diagrammatic view of an embodiment of a device according to the invention. FIG. 2 shows a further embodiment which is also suitable for ambulant treatment. FIG. 3 shows an embodiment which is suitable in particular for static treatment, for example in a clinic.

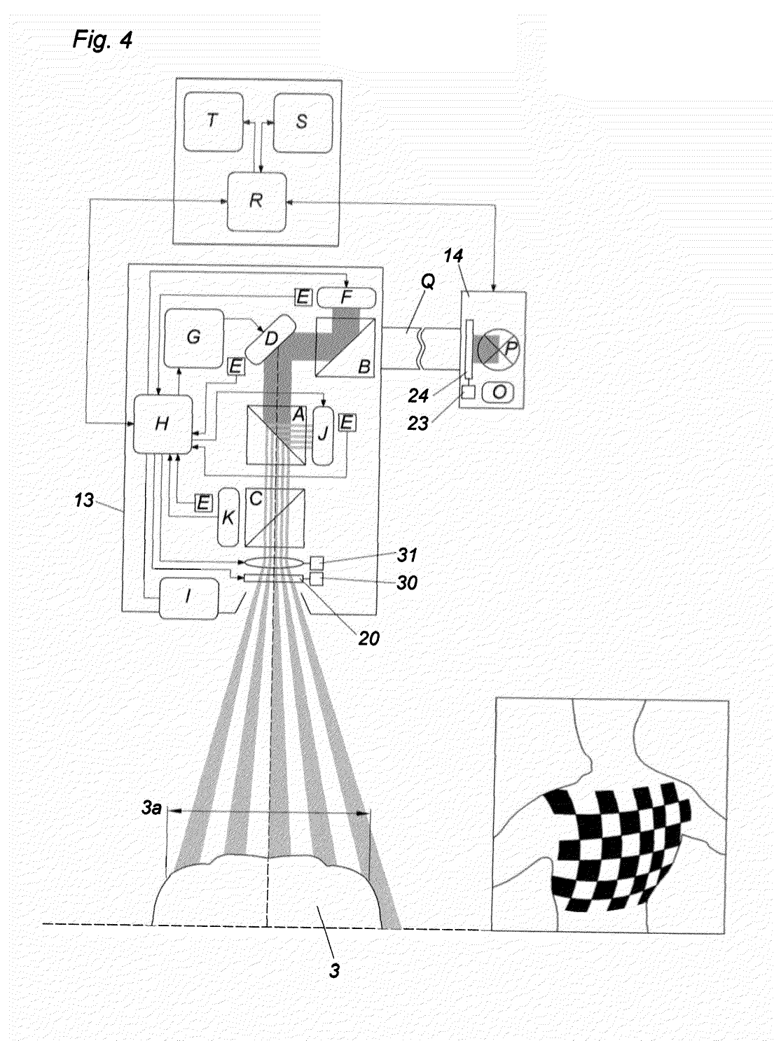

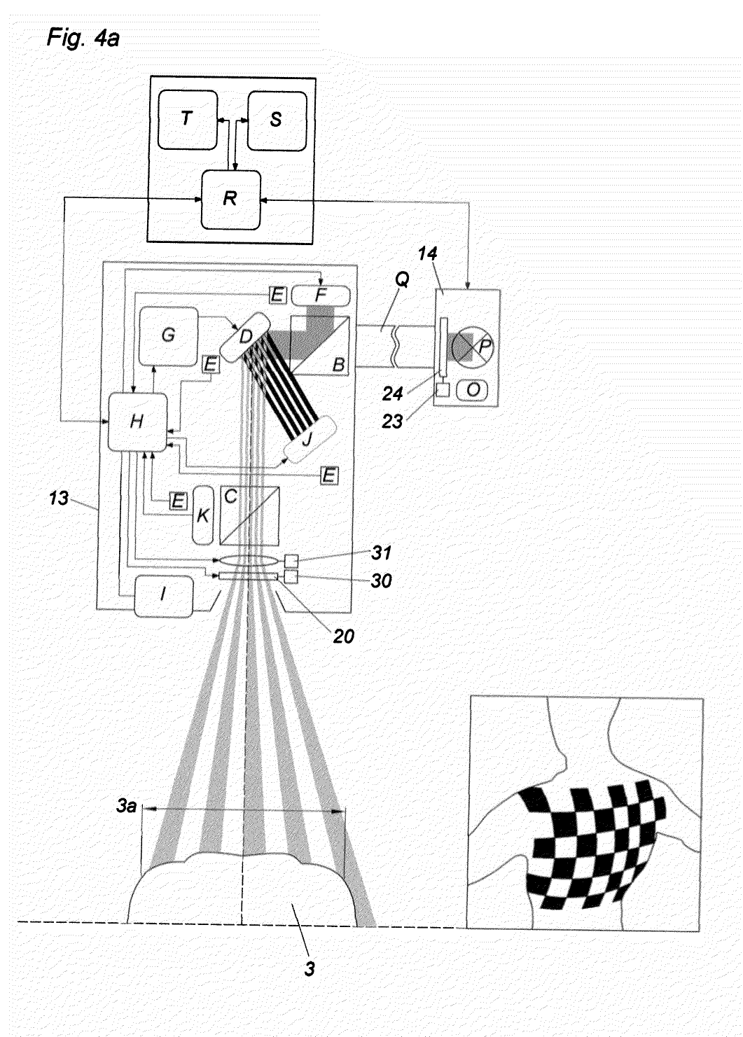

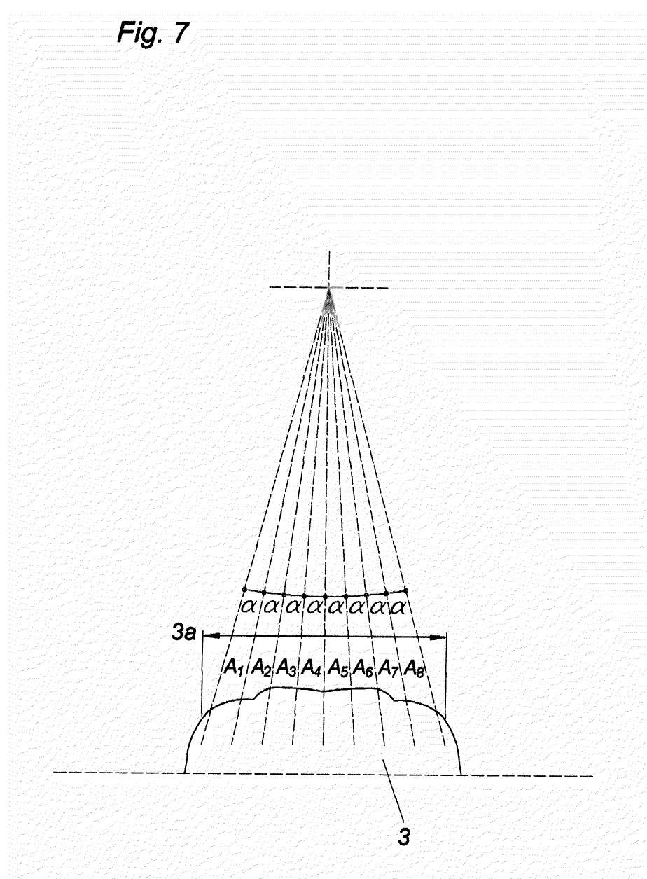

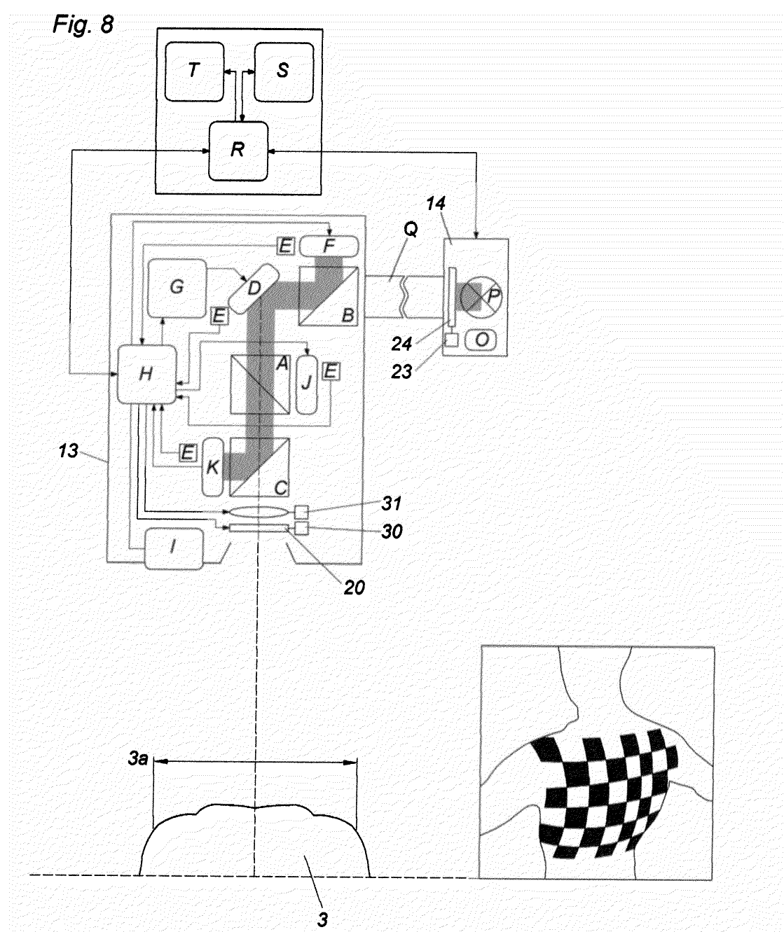

FIGS. 4, 5, 6, 8, 9, 10 and 11 show various operating conditions of a further embodiment of the invention as a diagrammatic view (with a LCOS modulator). FIG. 7 shows an explanatory view in regard to spatial detection of the configuration of the surface of the object, in particular the human skin. FIGS. 4a, 5a, 6a, 8a, 9a, 10a and 11a show various operating conditions of a further embodiment of the invention as a diagrammatic view corresponding to FIGS. 4, 5, 6, 8, 9, 10 and 11, but with a DLP or DMD modulator respectively.

The embodiment shown in FIG. 1 has a UV light source P which according to the invention is disposed outside the irradiation head 13 in a separate light source housing 14. Arranged between the light source housing 14 and the irradiation head 13 us at least one flexible optical waveguide Q, by way of which UV light from the UV light source P can be passed to the irradiation head 13.

The flexible optical waveguide can include at least one quartz glass fiber for the low-loss passage of UV light. To protect the flexible optical fiber it can be light-tightly sheathed.

To be able to more easily replace the individual components, in accordance with a preferred embodiment it can be provided that the flexible optical waveguide is connected by way of a releasable connection 15 to the UV light source housing 14 or the irradiation head 13.

The UV light is coupled into the optical waveguide by way of a coupling-in collimation optical system 16 and is coupled out in the irradiation head 13 by way of a coupling-out collimation optical system 17.

For control of the individual components, there is provided a control computer R which has a keyboard S or another input device, in particular a computer mouse and/or a light pen/graphics tablet etc. The control computer R has a display screen (DFD, plasma, CRD) or a holographic projector as the display device. In the present example shown in FIG. 1 the control computer is a laptop or a notebook.

A device which is preferably electronically actuable by way of the lines 18 is arranged in the irradiation head 13, for variably adjusting the light distribution at the object 3, more precisely the surface 3a of the object, that is to be irradiated. That device is only diagrammatically illustrated in FIG. 1 and is denoted by reference 19. With such a device which will be described in greater detail by means of the embodiments by way of example hereinafter, it is possible for subregions of the region 3a of the objective 3, that is to be irradiated, to be selectively irradiated with different levels of intensity, which is of great advantage for the treatment of various skin diseases because it is possible in that way for the radiation intensity to be well adapted to the local affliction. In that case the light passes out of the irradiation head 13 by way of the optical imaging system 20 which is only diagrammatically illustrated as a lens but which in practice can also include a plurality of lenses.

The irradiation head can further have a visible light-emitting light source F which is only diagrammatically shown in FIG. 1. By way of that light source, it is possible to project a visible image on the skin Furthermore, a camera, preferably an electrical image signal-delivering CCD camera K, can be arranged in the irradiation head 13. As is described in greater detail hereinafter, that camera can on the one hand receive light from the UV light source P or the colored light source F, which is relevant primarily for calibration purposes. In operation however the CCD camera K can also record images of the region 3a to be irradiated and acquire the UV light reflected by the surface 3a during the irradiation operation. That is described in greater detail hereinafter.

In addition a device I for detecting the spacing and/or the spatial configuration of the surface 3a of the object can be arranged on the irradiation head 13. By way of that device it is possible to exactly establish the levels of intensity actually passing into the subregions of the surface 3a. More specifically the intensity depends not only on the energy irradiated in a given solid angle region but also on the area of the subregion which is irradiated. That area in turn depends on the spacing and the spatial configuration of the surface of the object. If the geometrical configuration is known, then—as is described in greater detail hereinafter—the energy doses in the individual solid angle regions can be corrected in such a way that the desired intensity is actually produced on the surface to be irradiated. That even occurs dynamically, for example when the patient is breathing and thus the surface 3a is moving. In addition a carrier device generally identified by reference 21 for the irradiation head 13 is provided in FIG. 1. The radiation head 13 can be mounted displaceably and/or rotatably to the carrier device to achieve an optimum orientation in relation to the surface to be irradiated. It is also possible for the irradiation head to be displaceable by motor means.

A photospectrometer O supplied with light from the UV light source P by way of a beam splitter 22 can be provided in the light source housing 14 to be able to detect the spectral light distribution of the UV light of the UV source P.

Finally a closure shutter 24 which is preferably movable by way of a motor 23 can be provided in the light source housing. By way of the closure shutter, even when the UV light source P is switched on, it is possible to prevent light from issuing into the optical waveguide and thus the irradiation head when the UV light is not required there.

The UV light source housing 14 is connected overall to the control computer R by way of lines 25 which can also be combined together to form a bus line.

FIG. 2 shows an embodiment of a device according to the invention, which is suitable for an ambulant use. The same references denote the same parts as in FIG. 1. A region 3a can be established by way of the irradiation head. The size of the irradiation window g is afforded by way of the spread angles H. The spacing is identified by f.

The irradiation head 13 can be telescopically linearly displaced in respect of the height e. The irradiation head 13 can also be adjusted in the heightwise angle (arrow 26) and in the azimuth angle (arrow 27). Linear displacement in the horizontal direction (arrow 28) is also possible. Finally the irradiation head 13 can also be rotatable about the broken-line optical axis leading to the patient, preferably through 90°. A rectangular irradiation surface can thus be converted from an upright format to crosswise format (and vice-versa). In that way the treatment head 13 can be optimally oriented relative to the object (patient 3) who in the present example is sitting on a chair.

In the FIG. 3 embodiment the irradiation head 13 is also mounted displaceably to a carrier device 21. It has two linear axes displaceable by motor means in the vertical and horizontal directions. The rotary mounting of the irradiation head 13 can also be adjusted by motor means. Such adjustment is effected by way of the control computer R which is connected to the adjusting motors in a manner not shown here.

In contrast to the FIG. 2 embodiment, the FIG. 3 embodiment provides that the object or the patient himself is also movable, by standing on a turntable 29 actuated by the control computer R. Therefore not just the irradiation head but also the patient is moved for the relative orientation of the irradiation head 13 on the one hand and the patient 3 on the other hand.

In the following Figures identical references denote identical or equivalent parts, to the preceding Figures.

In the FIG. 4 embodiment the irradiation head 13 is shown on a larger scale. On the other hand optical details such as for example the optical collimation system which are not necessary to understand the invention are omitted for the sake of simplicity. Structurally, the configuration of the overall installation is similar to FIG. 1. There is a housing 14 for the UV light source P connected by way of a flexible optical waveguide Q to the actual irradiation head. The electronic components of the control computer R together with the keyboard S and the screen T are also arranged separately and are connected by way of lines or a bus system to the irradiation head 13 on the one hand and the UV light source housing 14 on the other hand.

By way of the beam splitter B (preferably a dichroitic prism), on the one hand light from the UV light source P by way of the optical waveguide Q and on the other hand light from a colored light source F can pass to the further components of the irradiation head or on to the object 3a respectively.

In the FIG. 4 embodiment the installation is in a positioning or teach-in mode.

In this case the closure shutter 24 of the UV light source P is closed or the UV light source is switched off. In return, the visible light-emitting light source F is switched on. This can involve an RGB unit which preferably includes light emitting diodes and which can emit both colored and also white light. Colored light, for example red light, is emitted for the present adjusting operation. The light source F is actuated by the electronic control unit (control computer R) by way of the (sub-)control unit arranged in the irradiation head 13, for example FGPA or DSP. A temperature monitoring sensor E monitors the temperature of the visible light-emitting RGB light source F. Light passes by way of the beam splitter B from the light source F on to the electronically actuable spatial light modulator D (EASLM). That modulator can be for example a liquid crystal on silicon unit (LCOS). The modulator D is actuated by the control unit H by way of an image data processing unit G. Depending on the respective actuation of the modulator D, depending on the respective polarisation, light reflected thereby either passes through the splitter prism A with a polarisation filter and on to the dichroitic prism C or on to a cooling element G which absorbs that light which is not intended to go to the prism C and thus on to the object to be treated.

With the light modulator D which like also further components can be monitored by means of temperature sensors E, it is possible for given fields on the object to be illuminated for example in a notional pixel raster, and more specifically with a variable brightness or intensity, while however others are not. Finally the modulator D forms the core component for the selective radiation of subregions on the object to be irradiated.

In the operating mode shown in FIG. 4 for the treatment setup, the modulator D is actuated in such a way that a relatively large checkerboard-like pattern is produced on the object (see FIG. 4 at bottom right). In that operating condition the exit shutter 30 is opened by way of the motor 31. The optical imaging system 20 can be preferably steplessly displaced under the control of the control unit H by motor means (m) for the implementation of a zoom and focus adjusting function. After adjustment of zoom and focus (visible by sharp imaging of the checkerboard pattern on the object) orientation of the irradiation head relative to the patient or the object can be effected in such a way that, in the active irradiation window, the trapezium and pincushion distortion, due to the generally curved configuration of the object, is minimally pronounced. The camera K and the 3D scanner I which are also described hereinafter are not active. This only involves pre-adjustment of the irradiation head relative to the patient.

After that pre-adjustment operation is concluded, all relevant adjustment parameters can then be stored, for example in a patient/treatment file in the control computer R. In a further session those data can then be called up again to permit rapid pre-adjustment.

FIG. 4a shows another embodiment in the same operating mode as FIG. 4. In contrast to the LCOS unit D which operates on a polarisation basis, the embodiment of FIG. 4a has a DLP unit (digital light processing). For example this can involve a digital micromirror device (DMD) disposed on a chip. Such a DMD chip has microscopically small mirrors distributed over the surface, the edge length of which can be of the order of magnitude in the region of 10 μm. Those mirrors can be adjusted in their orientation under electronic control, for example by electrostatic fields. By virtue of the inclination of the individual micromirrors on the DMD chip D either the light is reflected directly to the beam splitter C and further on to the patient or it is passed to the absorber J. Various brightness stages of the individual pixels can be produced by pulse width-modulated actuation of the mirrors. Otherwise the structure is the same as in the LCOS variant shown in FIG. 4.

As FIGS. 2 and 3 show, in practice the screen D is arranged in such a way that it is viewable by the viewer, for example the physician, like also the irradiating region of the object 3. In that way it is possible for the image presented on the screen to be viewed simultaneously with a correlated image on the object, which is produced by the colored light source F by way of the modulator, and that is of great advantage for control purposes.

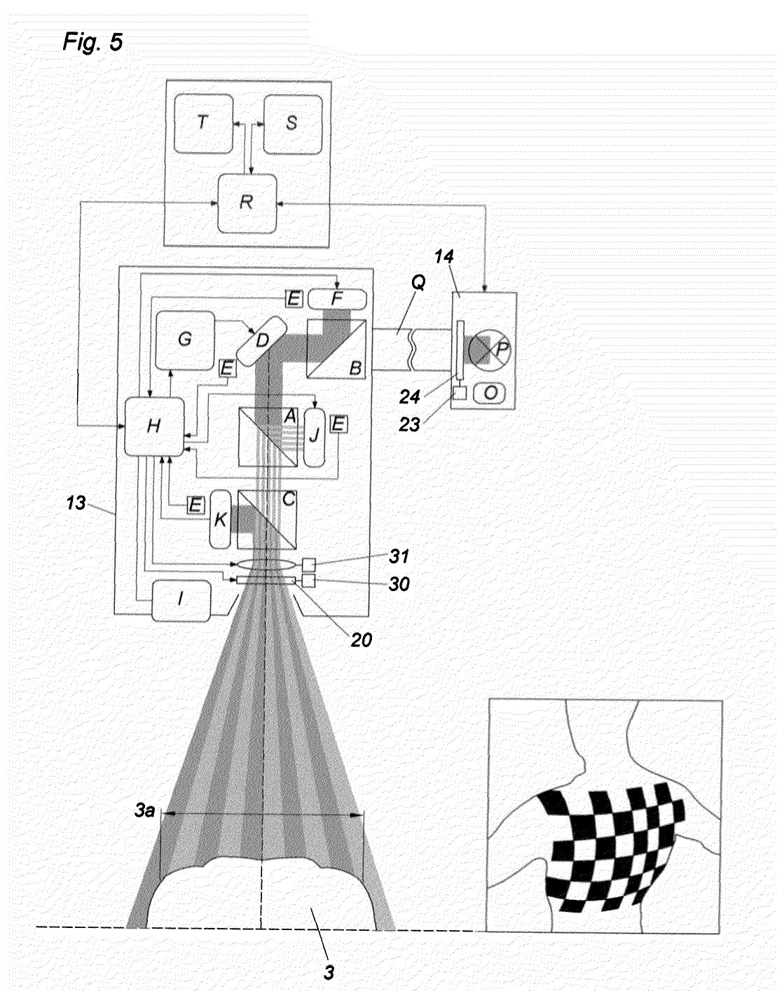

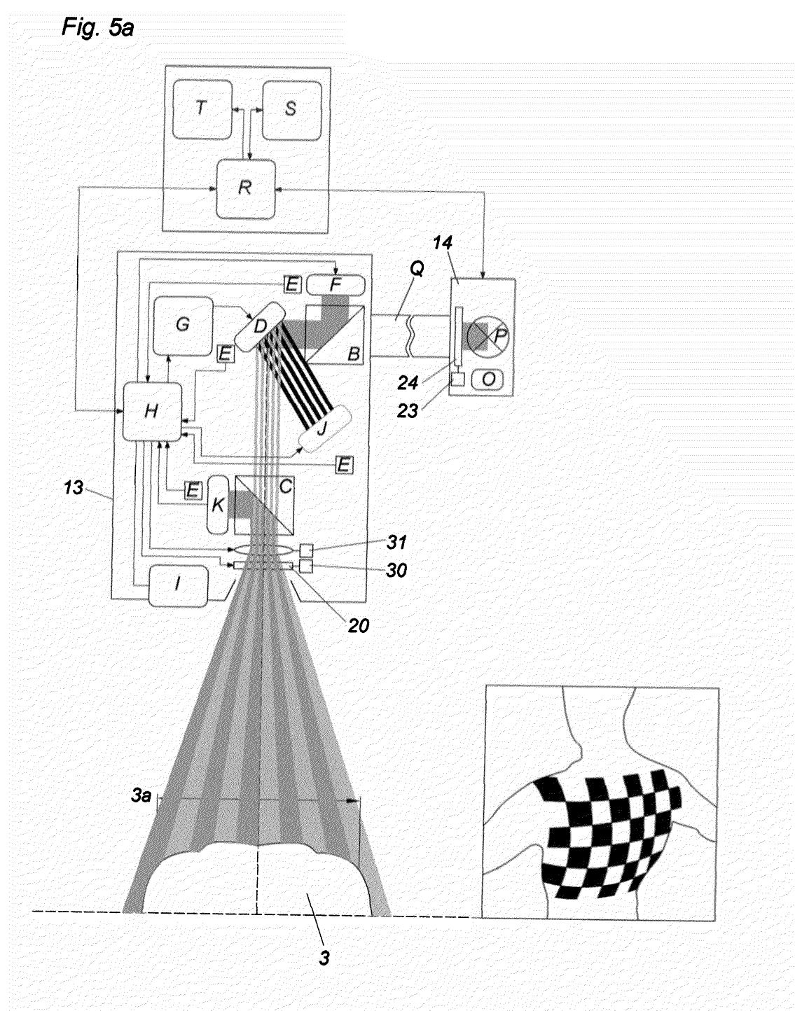

FIG. 5 shows the same device as FIG. 4, but in another operating mode—more specifically, for detecting an image of the region (3a) to be treated of the object during the next treatment setup step. For that purpose the device in the irradiation head 13 has a camera, preferably a CCD camera. That passes electrical image signals to the control unit H and further to the control computer R.

After positioning has been correctly concluded as shown in FIG. 4 the conclusion is confirmed on the control computer R by means of an operating or input element S. Thereafter the modulator D is automatically actuated in such a way that the light from the colored light source F is modified to give a regular pattern in the irradiation window or on the region 3a of the object 3, that is to be irradiated. Subsequently the CCD camera makes a recording of the projection pattern which is generally distorted because of the curvature of the surface 3a and which can be stored as a basis for the subsequent spatial imaging operation on the screen D.

FIG. 5a shows a variant of the invention shown in FIG. 5, in which a DMD unit D is used instead of the LCOS unit D, as in FIG. 4a.

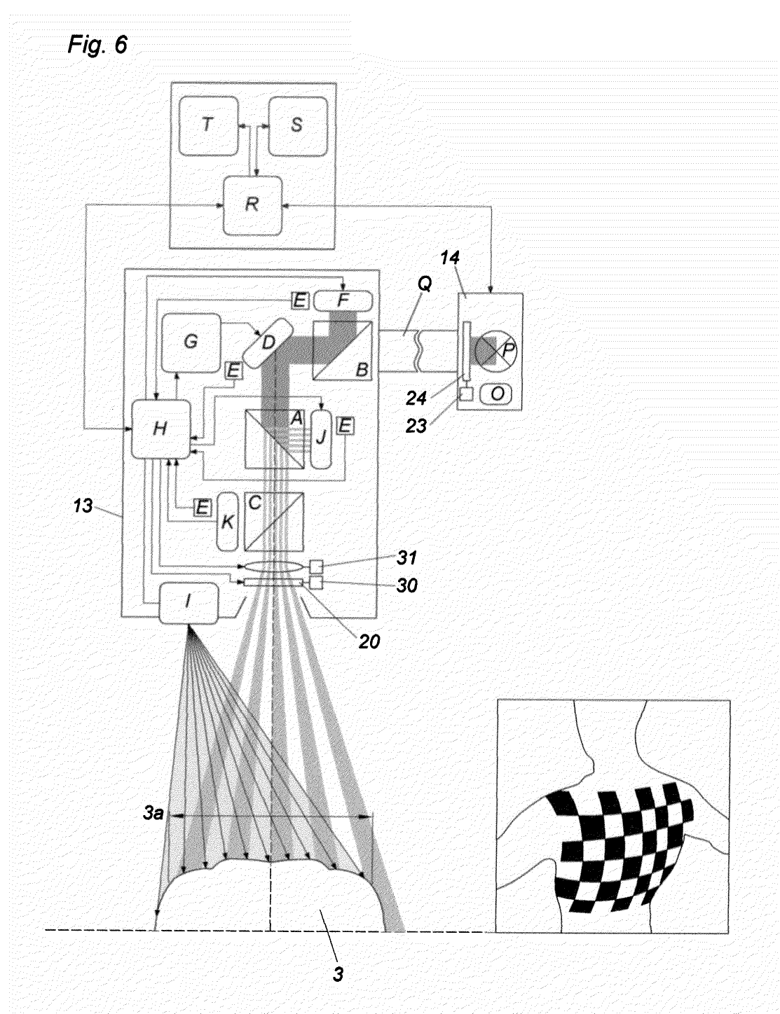

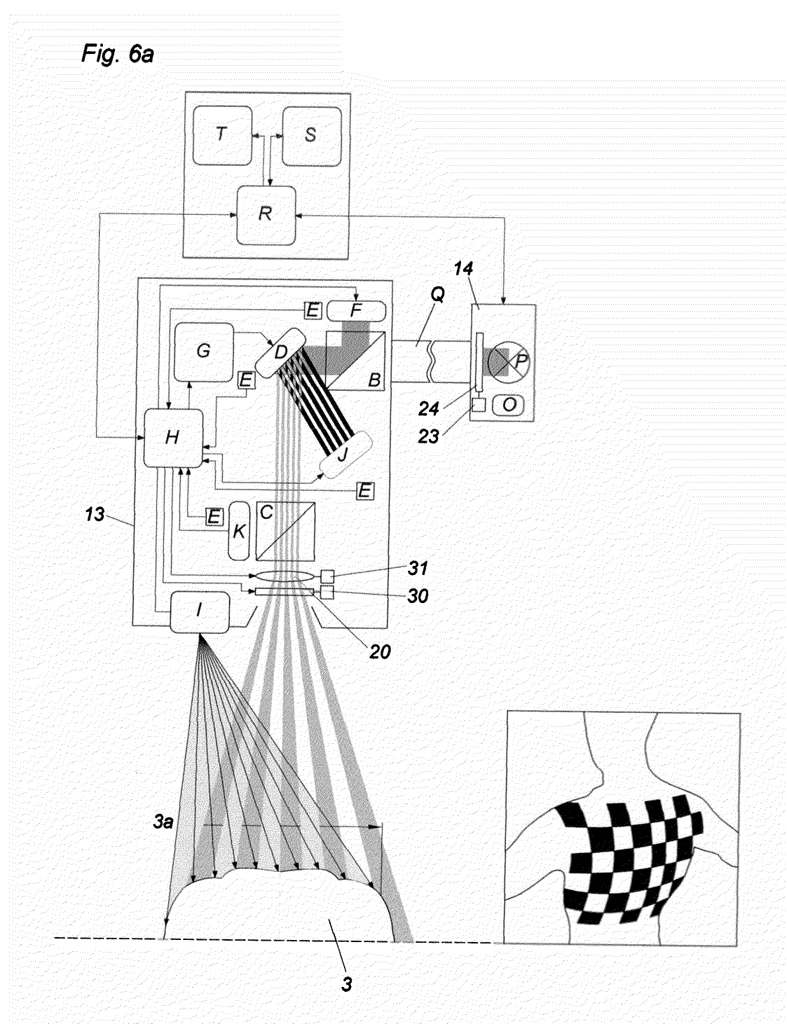

The method step shown in FIGS. 6 and 7 essentially involves taking account of and finally compensating by a computation procedure for the different sizes and positions of the individual irradiated subregion surfaces A1 through A7 (see FIG. 7), which are caused by virtue of the spatial structure of the surface 3a. If the energy delivered in a solid angle region a of a surface portion to be irradiated is known, then, to know the medically relevant intensity (that is to say energy per surface area and time), it is necessary to know the area of the individual subregions A1 through A7, which generally varies for each subregion, because it is generally at a different spacing from the irradiation head and also involves a different orientation or position.

In order to detect those individual surface portions A1 through A7 diagrammatically shown in FIG. 7, FIG. 6 provides a position detection device I for contactless detection of the spatial configuration of the region 3a, that is to be irradiated, of the surface of the object 3. The position detection device 3 is preferably arranged in or on the irradiation head 13 and measures therefrom the surface 3a. In a preferred embodiment the position detection device includes a 3D laser scanner for detecting the surface geometry of the object. The position detection device 3 may however also include a device for the projection of predefined patterns on to the object, which are then detected by a camera and electronically evaluated.

The position detection device I is activated by an electronic control device R which evaluates the measurement signals and possibly stores them.

Thus the 3D laser scanner I measures the surface region covered by the irradiation window and communicates its data to the control software in the control computer R by way of the control unit H. A spatial facet model of the surface region 3a covered by the optical imaging system 20 of the irradiation head 13 and the irradiation window is calculated. Together with the distorted image acquired by the CCD camera K as shown in FIG. 5 a 3D correction matrix is calculated by the control software, with a subregion of the surface to be irradiated or a corresponding solid angle region corresponding to each field or element of the matrix. The values in the 3D correction matrix are correlated with the position and size respectively of the surfaces A1 through A7 (see FIG. 7).

FIG. 6a again shows the DMD variant for the LCOS variant in FIG. 6.

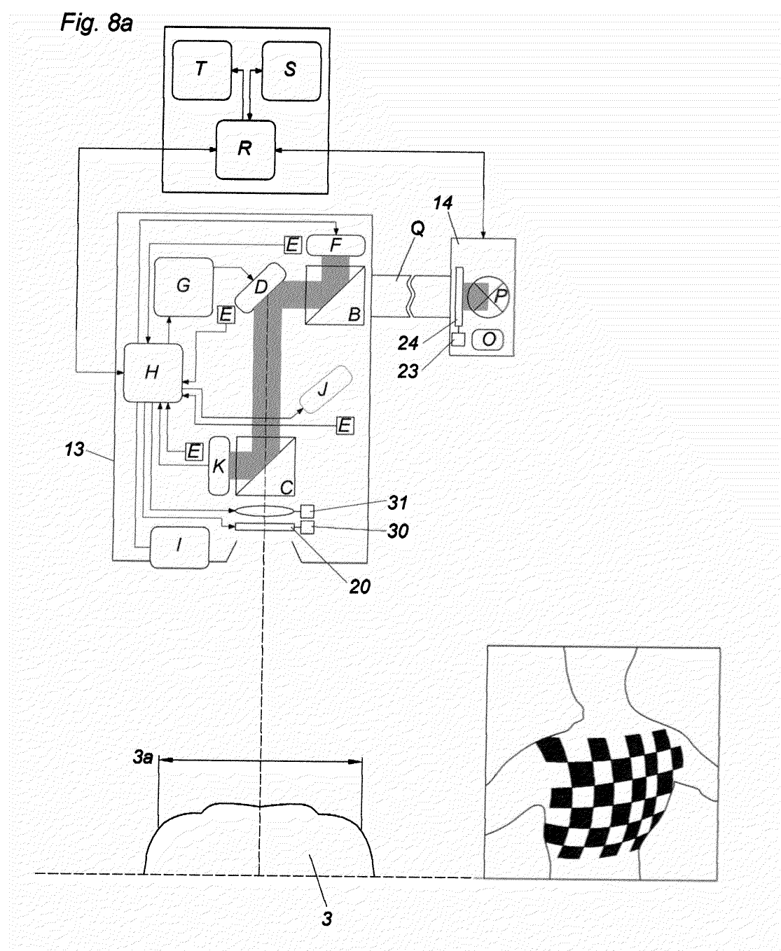

In accordance with the mode shown in FIG. 8, calibration of the RGB colored light source F and the CCD camera K can now be effected as the next step.

For that purpose the shutter 30 of the irradiation head 13 is closed by way of the motor 31 to be able to adjust the CCD camera K. The camera K communicates a dark image to the control unit H. The RGB unit F is then programmed to deliver white light. In that calibration step the prism C is pivoted through 90° (as is shown in FIG. 8) so that the light from the light source F passes by way of the modulator directly (that is to say not reflected by the object 3) on to the camera K. The camera K then sends an image to the control unit H which now calculates a correction matrix which is temporarily applicable over the treatment session, for possible image disturbances for dust or scratches. At the same time the control unit H calculates a correction matrix which optimises irregular illumination by the light source F by suitable correction modulation of the modulator D to afford a light distribution which is uniform over the projection window. In that step therefore irregularities in the light source F or other optical components can be compensated, stored and subsequently corrected.

FIG. 8a shows the DMD variant for the LCOS variant in FIG. 8.

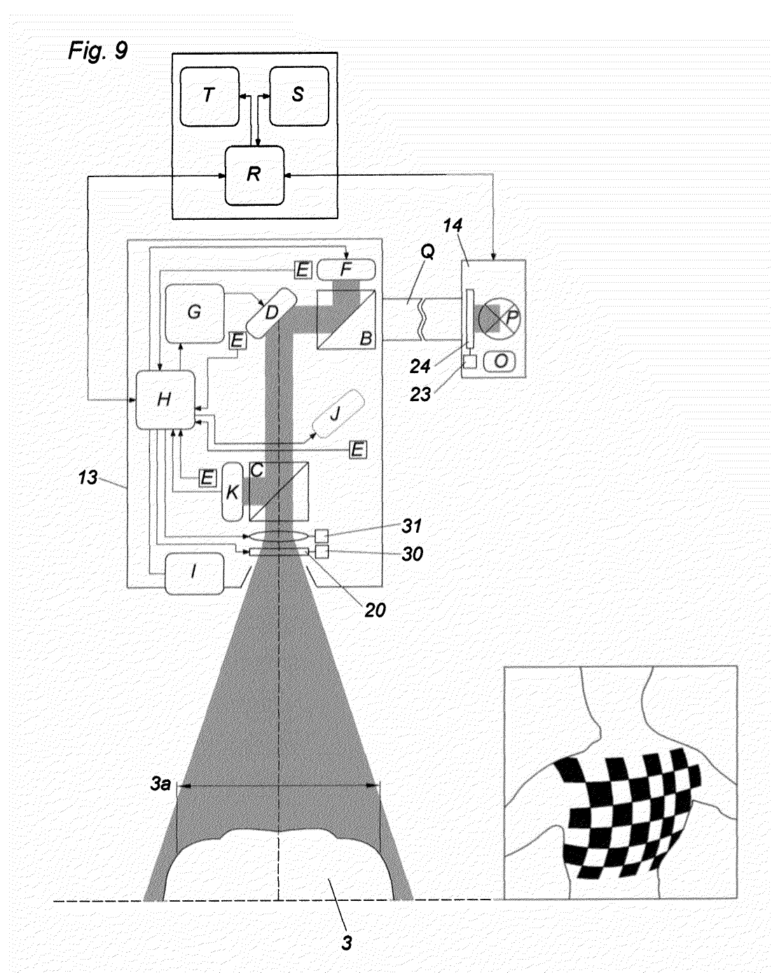

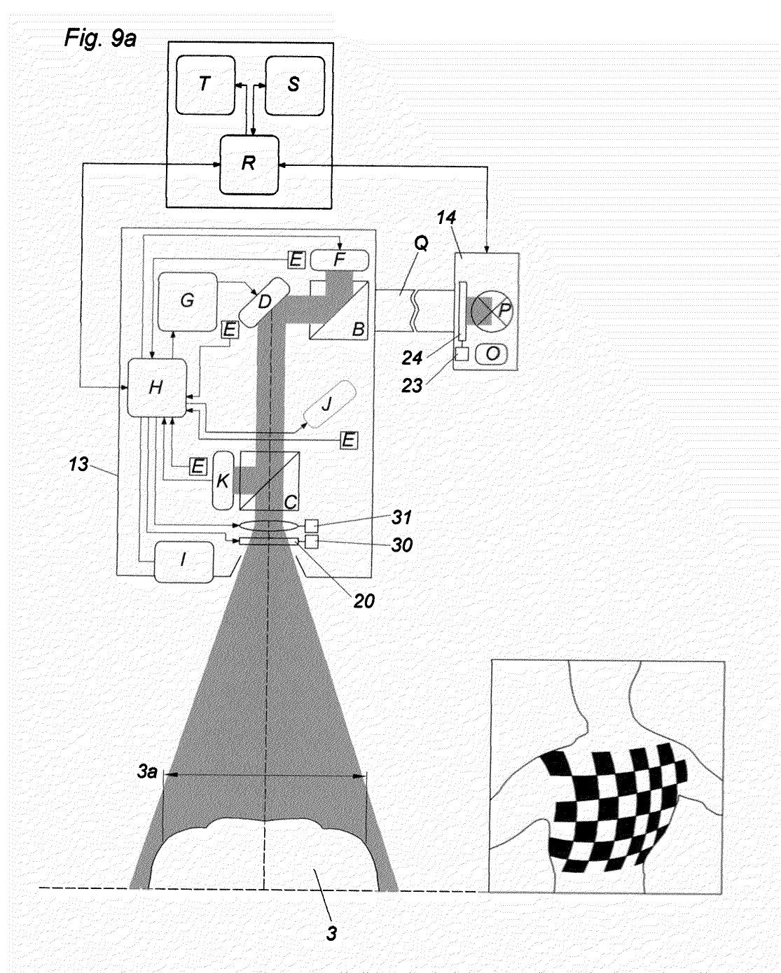

The mode shown in FIG. 9 involves visible image detection for the operator, for example for the physician. The irradiation head 13 by way of the RGB light source F and the modulator D delivers a white light, which is over the full surface area (and calibrated in accordance with the previous step), on to the irradiation surface 3a. With that illumination the camera K records for example a plurality of color images of the surface to be irradiated per second and communicates that stream of images by way of the control unit H to the control software in the control computer R. The light strength of the colored light source F can be so adjusted by means of the control software that a recording of the surface of the skin, which is exposed to light as well as possible and can thus be evaluated, is available within the control system for further processing.

FIG. 9a shows the DMD variant for the LCOS variant in FIG. 9.

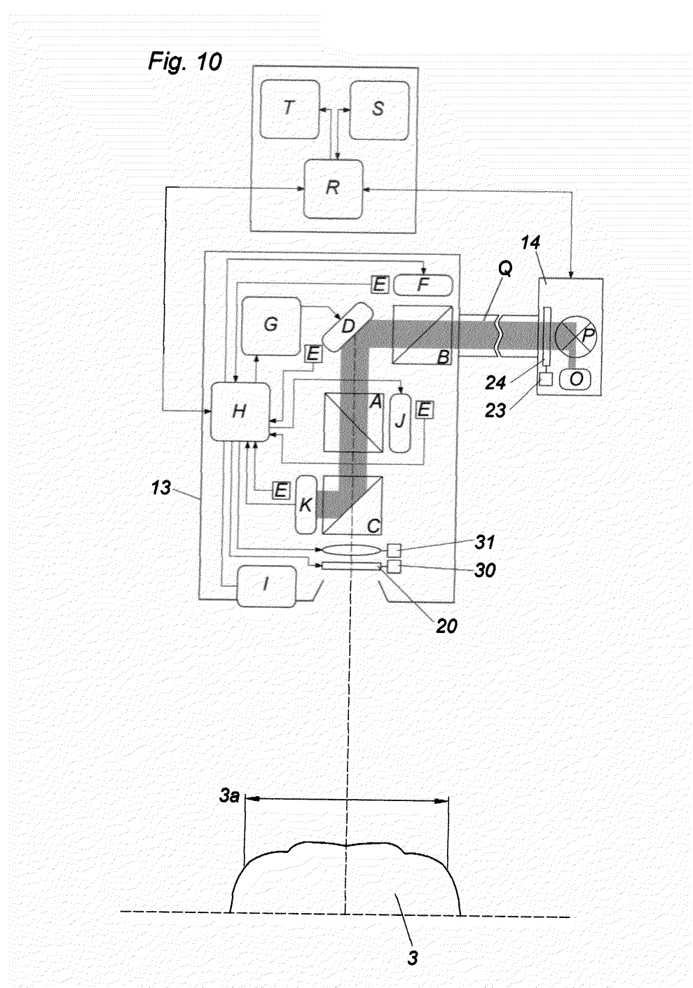

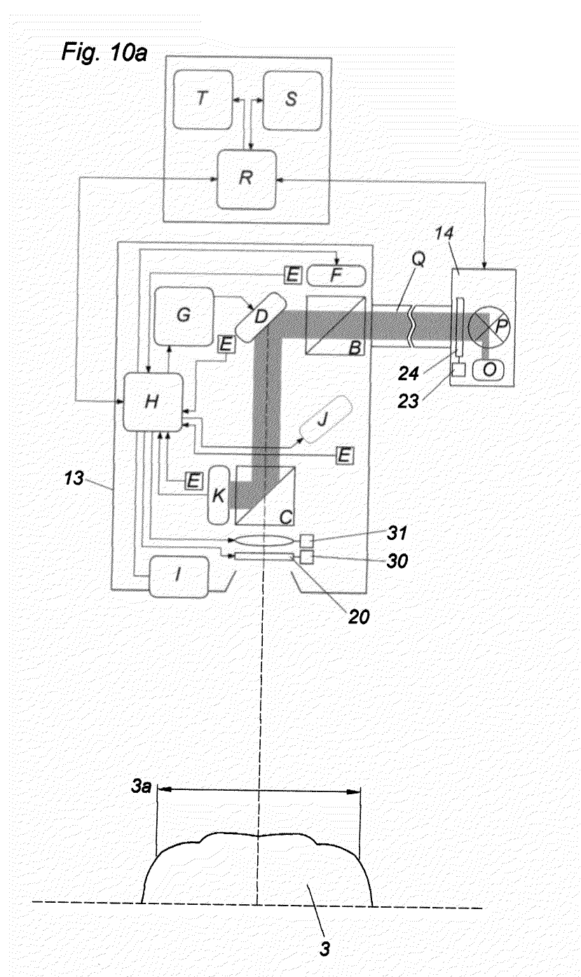

Referring to FIG. 10, before the commencement of treatment, all parameters are once again interrogated by the control software in the control computer for acknowledgement by the operator. The RGB light source F is now deactivated and the CCD camera K, by way of the control unit H, now takes over compensation in respect of the radiation intensity of the individual pixels (physical picture elements of the modulator D). For that the shutter 30 of the irradiation device is closed by way of the motor 31 and the shutter of the external UV light source P is opened by way of the motor 23.

The control software in the control computer R now alters the radiation intensity from 0% to 100% of the calculated maximum radiation intensity and the CCD camera sends those images to the control unit H. From all collected and stored items of image information, the control unit forms a two-dimensional correction mask (linearisation) in the form of a gray scale image which is so calculated with the previously defined medical irradiation mask (intensity reference values for the individual subregions on the object) that the correct modulation images correspond in the exact physical resolution of the modulator D by way of the modulation function (time/intensity) in the integral over each pixel to the predetermined irradiation dose.

Before the beginning of the actual treatment a check is also made by way of the photospectrometer O to ascertain whether the defined wavelength bandwidth is present.

FIG. 10a shows the DMD variant for the LCOS variant of FIG. 10.

Before the actual treatment—that is to say irradiation with UV light—begins, the physician or generally the operator has established the desired intensity reference values for the individual subregions of the object. That can be effected for example from patient data files which have been previously stored. It can however also be implemented directly on the screen, for example by painting thereon by means of a stylus. The physician does in fact have a visible image of the skin of the patient available on the screen and can easily identify the parts to be treated. By way of the RGB light source, in parallel therewith the region on the skin which is to be irradiated and which is identified by him on the screen can be projected on to the skin and thus checked at the same time.

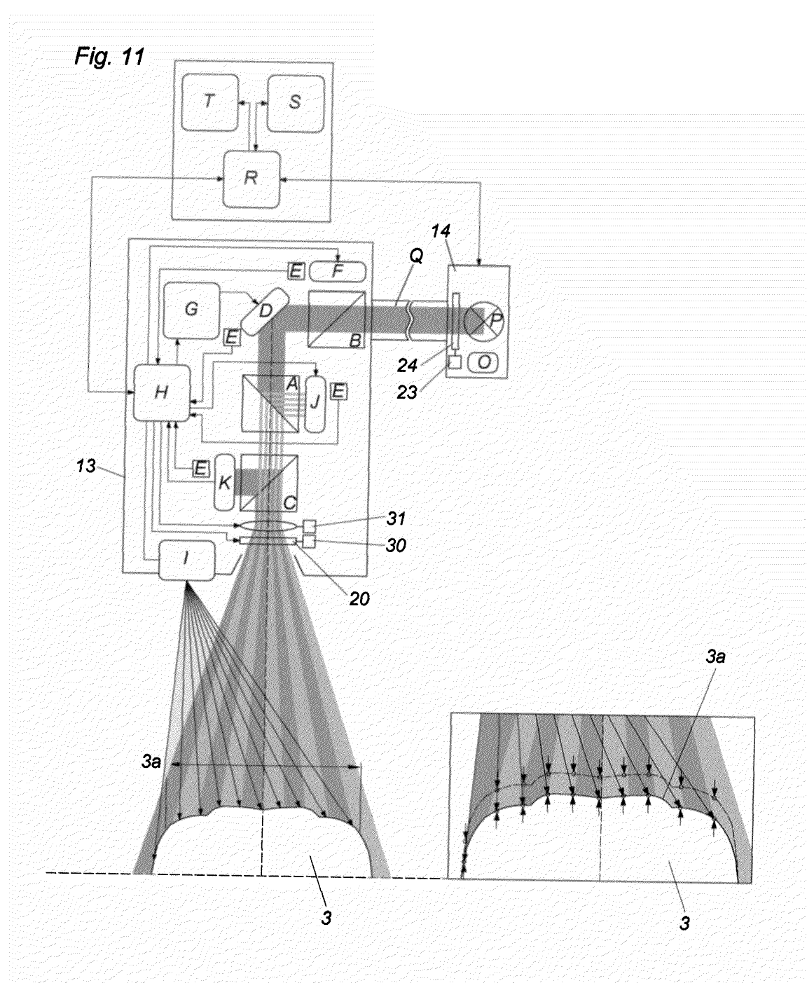

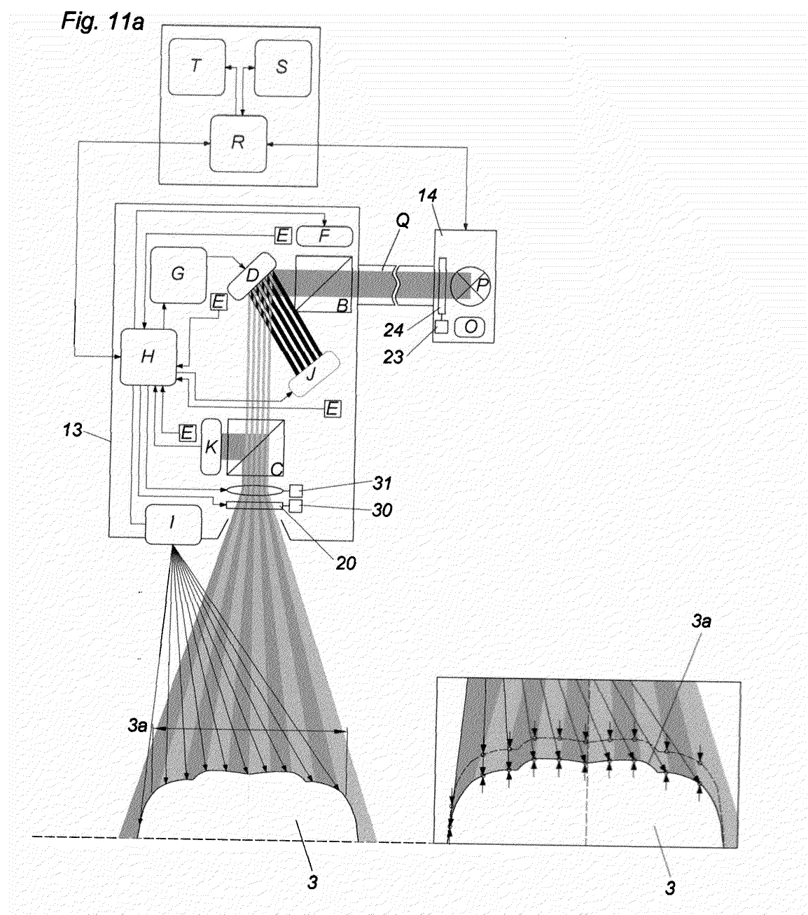

As the illustrated irradiation device, by way of the position detection device, always knows the position of the individual subregions, it is now possible by way of the control computer R or the control unit H to actuate the modulator D in such a way that the radiation power of the UV light delivered by the irradiation head into the solid angle region corresponding to the respective subregion, on the surface of the subregion of the object, substantially leads to the respectively desired intensity reference value. In other words: the physician or the operator does not need to concern himself about the position or the spacing of the object, not even when that changes for example due to respiration, as is diagrammatically shown at bottom right in FIG. 11. If for example a subregion of the surface, that is associated with a given solid angle region, moves away from the irradiation head and thus becomes larger in surface area, the modulator compensates for that by the supply of a correspondingly higher level of energy in that solid angle region so that the desired intensity reference value is achieved on the surface of the skin

FIG. 11a shows the DMD variant for the LCOS variant in FIG. 11.

The alternative irradiation procedure is shown in greater detail in FIG. 11, in which respect it will be seen that, in parallel with the UV light, the 3D laser scanner constantly monitors the position of the object.

It will be appreciated that the invention is not limited to the illustrated embodiments. Numerous modifications within the scope of the claims are conceivable and possible.

1. A device for irradiating an object, in particular the human skin, with UV light, comprising a UV light source and an irradiation head which includes an optical imaging system and from which UV light is projected on to the object, wherein a position detection device for contactless detection of the spatial configuration of the region of the surface of the object, that is to be irradiated.

2. A device as set forth in claim 1 wherein the position detection device is arranged in or on the irradiation head and measures therefrom the region to be irradiated of the surface.

3. A device as set forth in claim 1 wherein the position detection device includes a 3D laser scanner for detection of the surface geometry of the object.

4. A device as set forth in claim 1 wherein the position detection device includes a device for the projection of predefined patterns on to the object, a camera for detection of the image of said patterns and an evaluation device for ascertaining therefrom the spatial configuration of the surface of the object.

5. A device as set forth in claim 1 wherein an electronic control device, by way of which the position detection device can be activated and in which measurement signals originating from the position detection device can be evaluated and/or stored.

6. A device as set forth in claim 1 wherein a electronically actuated device for variable adjustment of the light distribution at the object is arranged in the irradiation head in such a way that different subregions of the region to be irradiated of the surface of the object can be irradiated with different intensity levels.

7. A device as set forth in claim 6 wherein the device for variable adjustment of the light distribution at the object includes an electronically actuatable modulator for spatial light (EASLM).

8. A device as set forth in claim 7 wherein the electronically actuatable modulator or spatial light (EASLM) has a digital micromirror device (DMD) or a liquid crystal on silicon unit (LCOS).

9. A device as set forth in claim 5 wherein the electronic control device actuates the device for variable adjustment of the distribution of light at the object for each subregion in dependence on inputted or stored intensity reference values for each subregion and in dependence on the position of the individual subregions, that is detected by the position detection device, in such a way that the radiation power of the UV light on the surface of the subregion of the object, that is delivered by the irradiation head in the solid angle region corresponding to the respective subregion, substantially leads to the respective intensity reference value.

10. A device as set forth in claim 6 wherein the total surface to be treated of the object is subdivided into mutually adjoining subregions which are preferably arranged grid raster-like.

11. A device as set forth in claim 9 wherein the electronic control device also controls the intensity of the UV light source.

12. A device as set forth in claim 1 wherein in addition to the UV light source there is provided a visible light-emitting light source, the light of which can be projected on to the object by way of the optical imaging system of the irradiation head.

13. A device as set forth in claim 12 wherein colored light and/or white light can be emitted by way of the visible light-emitting light source.

14. A device as set forth in claim 13 wherein the visible light-emitting light source includes an RGB unit preferably comprising light emitting diodes.

15. A device as set forth in claim 1, wherein the visible light-emitting light source is actuable by an electronic control unit, wherein the light intensity and/or the light color is adjustable.

16. A device as set forth in claim 1 wherein there is provided an electronic control device including a display screen and that a visible image correlated with the current screen representation can be projected on to the object by way of a device for variable adjustment of the light distribution at said object, said device being correspondingly actuated by an electronic control device.

17. A device as set forth in claim 16 wherein the screen and the region to be irradiated of the object are arranged in mutually juxtaposed relationship or in displaced relationship one behind the other in such a way that they can both be viewed from the same viewer position.

18. A device as set forth in claim 1, wherein a camera, preferably a CCD camera, is arranged in the irradiation head.

19. A device as set forth in claim 18 wherein the camera is so designed that it converts UV light emitted by the UV light source and/or visible light emitted by the visible light-emitting light source into corresponding electrical image signals.

20. A device as set forth in claim 19 wherein the camera directly detects light originating from the light source and/or light reflected by the object.

21. A device as set forth in claim 20 wherein there is provided an irradiation head, a change-over switching device, preferably a rotatable beam splitter, by way of which light directly originating from the light source or light reflected by the object can be selectively directed on to the camera.

22. A device as set forth in claim 1 wherein the UV light source is disposed outside the irradiation head in a separate light source housing and arranged between the light source housing and the irradiation head is at least one flexible optical waveguide, by way of which UV light from the UV light source can be passed to the irradiation head.

23. A device as set forth in claim 1 wherein the irradiation head is mounted displaceably on a carrier device.

24. A device for irradiating an object, in particular the human skin, with UV light, comprising a UV light source and an irradiation head which includes an optical imaging system and from which UV light is projected on to the object, including a visible light-emitting light source, an electronically actuable device for variable adjustment of the light distribution, a camera in the irradiation head and an electronic control device which, for calibration of the device, calculates a correction matrix from the image projected by the light source by way of the device on to the camera or the corresponding electrical image signals, stores the correction matrix and takes it into consideration upon irradiation of the object.

25. A device for irradiating an object, in particular the human skin, with UV light, comprising a UV light source and an irradiation head which includes an optical imaging system and from which UV light is projected on to the object, including an electronically actuable device for variable adjustment of the light distribution, a camera in the irradiation head and an electronic control device which calculates a respective correction matrix from images projected—preferably with different intensity levels of the UV light source—by the light source by way of the device on to the camera or the corresponding electrical image signals, stores the correction matrix and takes it into consideration upon irradiation of the object.

26. A device as set forth in claim 1 wherein the position detection device includes a TOF camera for detection of the surface geometry and the spacing relative to the surface of the object.

27. A device as set forth in claim 26 wherein the spacing relative to the surface can be measured by the TOF camera during irradiation with UV light by the irradiation head.

28. A method of measuring the surface geometry and the spacing relative to the surface of an irradiated object wherein the position detection device, preferably a TOF camera, detects the position of the surface of the object, that changes in spacing and orientation relative to the position detection device, during the irradiation of the object.

29. A method as set forth in claim 28 wherein the dosage of the UV light is adapted in dependence on signals from the position detection device directly to the surface of the object that changes in spacing and orientation.
